(19) United States
(12) Patent Application Publication
(10) Pub. No.: US 2015/0038541 A1
(43) Pub. Date: Feb. 5, 2015

(54) COMPOSITION AND METHODS FOR CELL MODULATION

(71) Applicant: APOSIGNAL BIOSCIENCE LLC, Birmingham, AL (US)

(72) Inventors: Jeffrey B. Smith, Birmingham, AL (US); Vincenzo Guarcello, Birmingham, AL (US)

(73) Assignee: APOSIGNAL BIOSCIENCE LLC, Birmingham, AL (US)

(21) Appl. No.: 14/385,271

(22) PCT Filed: Mar. 15, 2013
(86) PCT No.: PCT/US2013/032538

§ 371 Date: Sep. 15, 2014

Related U.S. Application Data
(60) Provisional application No. 61/612,490, filed on Mar. 19, 2012.

(51) Int. Cl. A61K 31/417; A61K 45/06

(52) U.S. Cl. CPC A61K 31/417; A61K 45/06
USPC 514/400; 435/375

(57) ABSTRACT

Composition and methods for affecting cell proliferation, metabolism or sensitization, including the potentiation of proteasome inhibitor therapy for the treatment of disease caused by deregulation of ubiquitin-proteasome systems, in a cell or subject. The composition includes a compound in combination with a ubiquitin-proteasome inhibitor. In one embodiment, the compound has the formula C42H35N5O3 and the ubiquitin-protease inhibitor is a proteasome inhibitor or an inhibitor of another component of the ubiquitin-proteasome pathway such as an E3 ubiquitin ligase. In one embodiment of the method, the compound is administered to a cell or subject and a therapeutically effective amount of a ubiquitin-protease inhibitor is also administered. In another embodiment, the compound is administered to a cell or subject to inhibit cell proliferation. In yet another embodiment, the compound is administered to a cell or subject to increase, or augment, glucose utilization.

### FIELD OF THE INVENTION

The present application relates to a composition and methods for affecting cell proliferation, metabolism or sensitization, including the potentiation of proteasome inhibitor therapy for the treatment of cancer.

### BACKGROUND OF THE INVENTION

The ubiquitin-proteasome pathway is a cellular system that regulates the degradation of proteins in a cell. Ubiquitin is a small protein that, when attached to other proteins, labels these proteins for degradation by the proteasome. The proteasome is an energy dependent compartmentalized protease, abundant in the cytosol and nucleus of mammalian cells. The proteasome consists of a barrel-shaped core particle and two distinct regulatory particles that form a “lid” and “base” of the barrel. The regulatory particles recognize a polyubiquitinylated protein, unfold the protein, and control the entry of the unfolded protein to the interior of the core, which catalyzes protein disassembly.

The ubiquitin-proteasome system disassembles most intracellular proteins including cell cycle and growth regulators, components of signal transduction pathways, enzymes of housekeeping and cell-specific metabolic pathways, and mutated or post-translationally damaged proteins. The system is also involved in processing major histocompatibility complex (MHC) class I antigens. Accordingly, the ubiquitin-proteasome system is involved in many basic cellular processes such as cell cycle and division, differentiation and development, the response to stress and extracellular modulators, morphogenesis of neuronal networks, modulation of cell surface receptors, ion channels and the secretory pathway, DNA repair, regulation of the immune and inflammatory response, biogenesis of organelles, and apoptosis (or genetically programmed cell death).

Deregulation of the ubiquitin-proteasome system is widely linked to human diseases due to its involvement in such fundamental cellular processes. In particular, the ubiquitin-proteasome system has been linked to different cancers. Cancers are a broad group of related diseases, all of which involve disregulated, or deregulated, cellular proliferation. Malignant neoplasms evolve from normal cells by a multistep developmental process that involves the acquisition of several fundamental biological capabilities (D. Hanahan and R. A. Weinberg, Cell 144, 646-674, 2011). These hallmarks of cancer cells are continuous proliferative signaling, evasion of growth suppressors, resistance to the physiological process of cell death, reprogramming of energy metabolism, evasion of immune surveillance, induction of angiogenesis, and activation of invasion and metastasis capabilities.

The proteasome itself has been a major target of cancer treatments because proteasome inhibitors kill cells by inducing genetically programmed cell death, i.e., apoptosis. Proteasome inhibitors block intracellular protein disassembly, and the destruction of cancer cells by proteasome inhibitors such as bortezomib is at least partly mediated by a family of cysteine proteases called caspases (Pop C, Salvesen G S., J. Biol. Chem. 284(33):21777-81, 2009). Caspases can be activated either by an extrinsic death receptor pathway or by an intrinsic pathway mediated by the Apaf-1:procaspase-9 apoptosome. The former pathway is triggered by external ligands such as TNF-alpha, TRAIL, Fas ligand, or other death receptor ligands. The death receptor ligand elicits the formation of a protein complex called DISC, which activates and includes initiator caspase-8. Subsequently DISC evokes the release of mitochondrial proteins including cytochrome c and Smac/DIABLO, which activate the intrinsic Apaf-1:procaspase-9 apoptosome. The latter pathway can be activated independently of the extrinsic pathway by a variety of agents that increase cell stress such as a variety of agents that damage genomic DNA. Irradiation and cancer chemotherapeutic agents generally induce apoptosis at least in part by damaging cellular DNA and activation of the intrinsic pathway of caspase activation.

Bortezomib is a commercially available protease inhibitor that has been investigated for the treatment of cancer. It is an N-protected dipeptide also known as Pyz-Phe-boroLeu,[1R)-3-methyl-1-({(2S)-3-phenyl-2-[(pyrazin-2-ylcarbonyl)amino]propanoyl}amino)butyl]boronic acid, PS-341, and marketed as Velcade™. The chemical formula of bortezomib is as follows:

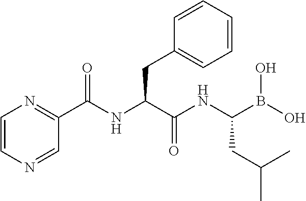 [SMILES: CC(C)C[C@H](NC(=O)[C@H](Cc1ccccc1)NC(=O)c1cnccn1)B(O)O]

Bortezomib is commercially available from Millennium Pharmaceuticals and other companies, such as BioVision, Inc. It was approved by the FDA in 2003 as a third-line therapy for refractory multiple myeloma, was extended to mantle cell lymphoma (non-Hodgkin lymphoma) in 2006, and became a frontline treatment for multiple myeloma in the U.S. in 2008 (King, J., Nature Biotechnology 28, 1236-1238, 2010). Bortezomib is the only proteasome inhibitor that has been FDA-approved, although several second-generation proteasome inhibitors are making progress in clinical trials (King, J., Nature Biotechnology 28, 1236-1238, 2010). Second generation proteasome inhibitors currently in clinical trials for solid tumors or hematologic cancers are: MLN 9708 (Takeda/Millennium); Carfilzomib ONX 0912 (Onyx Pharmaceuticals); CEP-1877 (Cephalon, Fraser, Pa., USA); NPI-0052 (Nereus Pharmaceuticals).

Bortezomib has been used successfully in the treatment of multiple myeloma. However, multiple myeloma often becomes refractory to all currently available chemotherapies. Multiple myeloma (also called myeloma) is the second most common type of blood cancer, after non-Hodgkin's lymphoma with approximately 20,000 new cases per year in the U.S.A. Multiple myeloma accounts for 1% of all cancers in white individuals and 2% in black individuals. There are three major limitations of bortezomib therapy for multiple myeloma: 1) the myeloma of the majority of patients is insensitive to bortezomib; 2) the myeloma eventually recurs and is refractory to bortezomib; and 3) the dose of bortezomib is limited by toxic side effects, most commonly peripheral neuropathy.

Peripheral neuropathy (PN) is the main dose-limiting toxicity associated with bortezomid therapy (Argyriou, A. A., et al., Blood 112(5):1593-9, 2008; Richardson, P. G., et al., Br. J. Haematol. 144(6):895-903, 2009). Grades 1 and 2 PN can occur in up to 75% and 33% of patients with recurrent or newly diagnosed disease under bortezomib therapy, respectively, whereas grades 3 and 4 neurotoxicity may affect up to 30% of patients with recurrent disease and up to 18% of patients with newly diagnosed disease. The onset of symptoms can be sudden, although they usually are cumulative during the course of therapy. Additionally the treatment is usually symptomatic with dose reduction and prolonging the interval between bortezomib infusions. For grade 3 or 4 neurotoxicity, sensory or motor loss can become disabling and cause bortezomib therapy to be suspended. The median time to improvement from the neurotoxicity is three months after discontinuation of bortezomib treatment. Accordingly, there are severe side effects associated with using proteasome inhibitors such as bortezomib.

In addition to the toxic side effects associated with bortezomib, several phase I and II clinical trials demonstrated that bortezomib has quite limited activity against solid tumors when used as a single agent or in combination with several anticancer drugs (Orlowski et al., Clinical Cancer Res. 14:1649-1657, 2008; Dick L. R. and Fleming P. E., Drug Discovery Today 15(5/6), 243-249, 2010). For example, bortezomib was able to inhibit proteasome activity and reduce the circulating level of interleukin-6 (IL-6), but showed limited clinical activity against metastatic breast cancer when used as a single agent (Yangl, C. H. et al., Annals of Oncology 17: 813-817, 2006). A phase I study of bortezomib in combination with weekly paclitaxel in patients with advanced solid tumors of the breast, ovaries, or prostate failed to show enhanced anti-tumor activity relative to that of paclitaxel alone (Cresta S., et al., Eur. J. Cancer 44:1829-1834, 2008). Similar results were found in patients with lung and head & neck cancer treated with bortezomib and cetuximab. No patient achieved a complete or partial response, and only six patients experienced stable disease for at least 12 weeks (Dudek A. Z., et al., Br. J. Cancer 100:1379-1384, 2009). No objective responses were observed in patients with advanced solid tumors treated with bortezomib, etoposide and carboplatin, however stable disease was noted in some highly refractory patients (Lieu, C., et al., Invest. New Drugs 27:53-62, 2009). Further, phase II studies did not find objective responses with bortezomib treatment in renal cell, colon, neuroendocrine tumors, or melanoma despite clear documented proteasome inhibition in tumor biopsies and peripheral blood lymphocytes. See, Davis, N. B., et al., J. Clin. Oncol. 22:115-119, 2004; Shah, M. H., et al., Clin. Cancer Res. 10:6111-6118, 2004; Markovic, S. N., et al., Cancer 103:2584-2589, 2005; and Mackay, H., et al., Clin. Cancer Res. 11:5526-5533, 2005.

Bortezomib and other second generation proteasome inhibitors aim to treat cancer via the targeting of the proteasome itself, however, other constituents of the proteasome-ubiquitin pathway can be targeted for the treatment of disease. A class of drugs closely related to the proteasome inhibitors target certain E3 ubiquitin (Ub) ligases and have also been investigated for the treatment of cancer. These drugs include MLN 4924 (Millennium), which targets Nedd8-activating enzyme (King, J., Nature Biotechnology 28, 1236-1238, 2010), JNJ-26854165 (Johnson & Johnson), and RG7112 (Roche), which targets hDM2 (King, J., Nature Biotechnology 28, 1236-1238, 2010).

The use of ubiquitin-proteasome inhibitors as therapeutic agents is also not limited to cancer treatment. Ubiquitin-proteasome inhibitors are under development for treating a variety of other proliferative diseases such as benign prostatic hyperplasia, and diseases such as rheumatoid arthritis, inflammatory bowel disease, lupus, cachexia, and autoimmune diseases. However, as noted above, the effectiveness of ubiquitin-proteasome inhibitors as therapeutic agents, particularly for anti-cancer treatment, is inconsistent.

Accordingly, there is a considerable need for a safe and effective composition and method for sensitizing cells to the modulation of a ubiquitin-proteasome pathway in a subject, and more particularly, potentiating ubiquitin-proteasome inhibitor therapy in a subject. In particular, there is a great need for therapies for the treatment of cancer such as multiple myeloma.

### SUMMARY OF THE INVENTION

A composition and methods for affecting cell proliferation, metabolism or sensitization, including the potentiation of proteasome inhibitor therapy for the treatment of disease caused by deregulation of ubiquitin-proteasome systems in a subject, are provided. The composition includes one or more ubiquitin-protease inhibitors in combination with a compound having the formula:

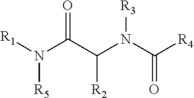

wherein R₁ and R₅ are each, independently, —H, a substituted or unsubstituted alkyl, a substituted or unsubstituted aryl, substituted or unsubstituted aralkyl or a substituted or unsubstituted heteroaralkyl; R₂ is —H, a substituted or unsubstituted alkyl, a substituted or unsubstituted aryl, substituted or unsubstituted aralkyl or a substituted or unsubstituted heteroaralkyl; R₃ is a substituted or unsubstituted alkyl, a substituted or unsubstituted aryl, a substituted or unsubstituted heteroaralkyl, or a substituted or unsubstituted heterocycloaralkyl; and R₄ is unsubstituted alkyl, a substituted or unsubstituted aryl, substituted or unsubstituted aralkyl, a substituted or unsubstituted cycloalkylalkyl, a substituted or unsubstituted heteroaryl, a substituted or unsubstituted heteroaralkyl, or a substituted or unsubstituted benzophenonyl; or an active derivative thereof.

In one embodiment, one of R1 and R5 is H and the other is a substituted or unsubstituted phenyl, dicycloalky-C1-C4-alkyl, branched C1-C12-alkyl, cyclic C3-C12-alkyl, phenyl-C1-C4-alkyl. Suitable identities for R1 or R5 include, but are not limited to, 1,2-diphenylethyl, 2-(4-ethylphenyl)ethyl, benzyl, 2,2-diphenylethyl, 2-cyclopentyl-2-phenylethyl, 2-phenyl-2-piridylethyl, diphenylmethyl, 3,3-diphenylpropyl, 3,4,5-tri-methoxybenzyl, 2,4,4-trimethylisopentyl, and 2-(4-methoxyphenyl)ethyl. In other or further embodiments, R2 is a substituted or unsubstituted phenyl, heteroaryl-C1-C4-alkyl, phenylfuranyl, diphenyl-C1-C4-alkyl, and phenyl-C1-C4-alkyl. Suitable identities for R2 include, but are not limited to, phenyl, 2-cyanophenyl, 3-cyanophenyl, 4-cyanophenyl, diphenylmethyl, pyrazolylmethyl, 2,4-dimethylphenyl, 2-methylphenyl, 3-methylphenyl, 4-methylphenyl, 2-methyl-4-methoxyphenyl, 3-methyl-4-methoxyphenyl, 4-methylthiophenyl, 3-chlorophenyl, 3-triflouromethylphenyl, benzyl, 2-trifluoromethylbenzyl, 3-trifluoromethylbenzyl, 2-chlorobenzyl, 3-chlorobenzyl, 4-chlorobenzyl, 2-methoxybenzyl, 3-methoxybenzyl, 4-methoxybenzyl, 2-flurobenzyl, 3-fluorobenzyl, 4-fluorobenzyl, 3-azidylphenyl, and 5-phenylfurna-2-yl. In other or further embodiments, R3 is an alkyl substituted with a heterocylcoalkyl group. Suitable identities for R3 include, but are not limited to, 2-(imidazol-4-yl)ethyl, 3-(imidazol-4-yl)propyl, 3-(imidazol-1-yl)propyl, 2-(3-methylimidazol-4-yl)ethyl, 2-(morpholine-4-yl)ethyl, 2-(4-pyrazol)ethyl, 4-pyrazolylmethyl, 2-N,N-dimethylaminoethyl, 3-N,N-dimethylaminopropyl, and 2-(aminocarbonyl)phenyl. In other or further embodiments, R4 is a substituted or unsubstituted phenyl. Suitable identities for R4 include, but are not limited to, benzophenon-2-yl, 4′-methoxybenzophenon-2-yl, 4′-chlorobenzophenon-2-yl, 2-(furan-2-yl)phenyl, 2-(thiophen-2-yl)phenyl, 2-benzylphenyl, 2-pyridylcarbonylphenyl, 2-(phenoxymethyl)phenyl, 2-(t-butylcarbonyl)phenyl, 2,2-diphenylethyl, 1-fluorenyl, (naphtha-2-yl)methyl, naphtha-1-yl, 3-(phenylcarbonyl)propyl, 4-phenylbutyl, 2-(4-chlorophenylcarbonyl)phenyl, 4-butylphenyl, 2-(4-chlorophenylcarbonyl)phenyl, 3-methoxyphenyl, N-methylpyrrol-2-yl, 2,3-dimethoxyphenyl, 3-butyl-2-pyridyl, 2-naphthylmethyl, 2-cyclohexylethyl, N-methyl-2-pyrrolyl, 2-cyclopentylethyl, 3-oxobutyl, 2-benzopyrazyl, quinoxalin-2-yl, 3-idolyl, (2-methylindol-3-yl)methyl, 3-(indol-3-yl)propyl, (indol-3-yl)methyl, (5-bromoindol-3-yl)-1-hydroxyethyl, 3-fluorophenyl, 1-phenyl-1-hydroxymethyl, 2-phenylphenyl, 2-phenoxyphenyl, thiophen-2-yl, and isopropyl.

In another embodiment, the compound is a ubiquitin-proteasome modulation sensitizing compound. In this embodiment, the compound is present in the composition in an amount effective to potentiate the proteasome inhibitor.

In another embodiment, the compound, designated herein as AS-7, has the molecular formula C₄₂H₃₅N₅O₃ and the following chemical structure:

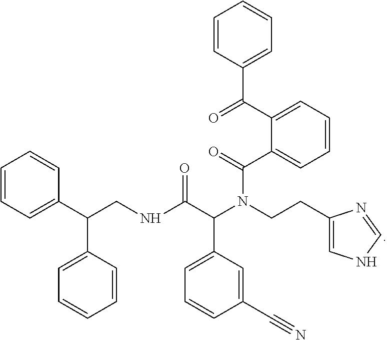 [SMILES: N#Cc1cccc(C(C(=O)NCC(c2ccccc2)c2ccccc2)N(CCc2c[nH]cn2)C(=O)c2ccccc2C(=O)c2ccccc2)c1]

The ubiquitin-protease inhibitor can be a proteasome inhibitor or an inhibitor of another component of the ubiquitin-proteasome pathway such as an E3 ubiquitin ligase. Ubiquitin-proteasome inhibitors include, but are not limited to, bortezomib, carfilzomib, disulfiram, epigallocatechin-3-gallate, salinosporamide, lactacystin, clasto-beta-lactone, epoxomicin, eponemycin, dihydroeponemycin, MLN 519, MLN 9708, ONX 0912, CEP-1877, NPI-0052, NLVS (4-hydroxy-5-iodo-3-nitrophenylacetyl-Leu-Leu-leucinal-vinyl sulfone), MLN 4924, JNJ-26854165, and RG7112.

In accordance with one of the methods, the compound is administered to a cell or subject and a therapeutically effective amount of a ubiquitin-protease inhibitor is also administered to the cell or subject. The compound is administered concurrently with, after, before or between cycles of administration of the proteasome inhibitor. Use of the method results in the potentiation of the ubiquitin-protease inhibitor, and thereby, improves the efficacy of the ubiquitin-proteasome inhibitor. Accordingly, the method improves the efficacy of treatments for cancer, rheumatoid arthritis, autoimmune disease, transplant rejection, multiple sclerosis, sepsis, inflammatory bowel disease, lupus, proliferative disease, benign tumors, hyperplasias, and cachexia. The method is particularly useful for treatment of the multiple myeloma.

In accordance with another one of the methods, an effective amount of the compound is administered to a cell or subject to inhibit cell proliferation.

In accordance with yet another one of the methods an effective amount of the compound is administered to a cell or subject to increase, or augment, glucose utilization.

### BRIEF DESCRIPTION OF THE DRAWINGS

FIG. 1 illustrates that AS-7 potentiates the cytotoxic action of anticancer drug bortezomib in HeLa cells. These data are mean±SD, n=3. Values are relative to controls that contained only the vehicle in which the drugs were dissolved.

FIG. 2 illustrates that AS-7 potentiates the cytotoxic action of anticancer drug bortezomib in multiple myeloma cells. These data are mean±SD, n=3. Values are relative to controls that contained only the vehicle in which the drugs were dissolved.

FIG. 3 illustrates that AS-7 potentiates cytotoxic action of the proteasome inhibitor NLVS in HeLa cells. These data are mean±SD, n=3. Values are relative to controls that contained only the vehicle in which the drugs were dissolved.

FIG. 4 illustrates that AS-7 has no effect on the cytotoxic activity of the anticancer drug paclitaxel. These data are mean±SD, n=3. Values are relative to controls that contained only the vehicle in which the drugs were dissolved.

FIG. 5 illustrates that AS-7 has no significant effect on the cytotoxic activity of the anticancer drug doxorubicin. These data are mean±SD, n=3. Values are relative to controls that contained only the vehicle in which the drugs were dissolved.

FIG. 6 illustrates that AS-7 potentiates the cytotoxic action of anticancer drug bortezomib in ovarian tumor derived IGROV cells. These data are mean±SD, n=3. Values are relative to controls that contained only the vehicle in which the drugs were dissolved.

FIG. 7 illustrates that AS-7 potentiates the cytotoxic action of anticancer drug bortezomib in ovarian tumor derived A2780 cells. These data are mean±SD, n=3. Values are relative to controls that contained only the vehicle in which the drugs were dissolved.

FIG. 8 illustrates that AS-7 potentiates the cytotoxic action of anticancer drug bortezomib in melanoma A2620 cells. These data are mean±SD, n=3. Values are relative to controls that contained only the vehicle in which the drugs were dissolved.

FIG. 9 illustrates that AS-7 potentiates the cytotoxic action of anticancer drug carfilzomib in HeLa cells. These data are mean±SD, n=3. Values are relative to controls that contained only the vehicle in which the drugs were dissolved.

FIG. 10 illustrates the concentration dependence of the inhibition of HeLa cell growth by AS-7. These data are mean±SD, n=3. Values are relative to controls that contained only the vehicle in which the drugs were dissolved.

FIG. 11 illustrates that AS-7 decreases the growth rate of HeLa cells. The time course data for the HeLa cell line were fit by nonlinear regression analysis to the equation for exponential growth using GraphPad Prism software.

FIG. 12 illustrates that AS-7 decreases the growth rate of melanoma A2620 cells. The time course data for the A2620 cell line were fit by nonlinear regression analysis to the equation for exponential growth using GraphPad Prism software.

FIG. 13 illustrates that AS-7 decreases the growth rate of ovarian A2780 cells. The time course data for the A2780 cell line were fit by nonlinear regression analysis to the equation for exponential growth using GraphPad Prism software.

FIG. 14 illustrates that AS-7 decreases the growth rate of breast T47D cells. The time course data for the T47D cell line were fit by nonlinear regression analysis to the equation for exponential growth using GraphPad Prism software.

FIG. 15 illustrates that AS-7 evokes accumulation HeLa cells in the G₀-G₁ phase of the cell cycle. HeLa cells were harvested two days after treatment with or without AS-7, stained with propidium iodide and subjected to analysis by FACS (Fluorescence Activated Cell Sorter).

FIG. 16 illustrates that AS-7 evokes accumulation of breast T47D cells in the G₀-G₁ phase of the cell cycle. T47D cells were harvested one, two, or three days after treatment with or without AS-7, stained with propidium iodide and subjected to analysis by FACS (Fluorescence Activated Cell Sorter).

FIG. 17 illustrates that AS-7 blocks exit from the Go-G1 phase of the cell cycle. HeLa cells were treated with nocodazole which evoked accumulation of the cells in the G₂-M phase of the cell cycle (Nocodazole Blocked). The cells were released from the nocodazole block and treated with or without AS-7. After 16 hours the cells were stained with propidium iodide and subjected to analysis by FACS (Fluorescence Activated Cell Sorter).

FIG. 18 illustrates that AS-7 fails to antagonize TNF alpha in HeLa cells. The time course data for the HeLa cell line were fit by nonlinear regression analysis to the equation for exponential growth using GraphPad Prism software.

FIG. 19 illustrates that AS-7 increases glucose utilization and acid production in HeLa cells. HeLa cells were cultured (100 mm diameter plates) in medium containing 4.5 mg/ml glucose at pH 7.4. After 5 days medium glucose was determined by standard methodology and the pH of the medium was measured with a pH meter.

### DETAILED DESCRIPTION OF THE INVENTION

A composition and methods for affecting cell proliferation, metabolism or sensitization, including the potentiation of proteasome inhibitor therapy for the treatment of disease caused by deregulation of ubiquitin-proteasome systems in a subject, are provided. The composition includes a ubiquitin-protease inhibitor in combination with a compound having the formula:

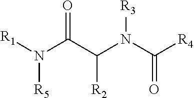

wherein R₁ and R₅ are each, independently, —H, a substituted or unsubstituted alkyl, a substituted or unsubstituted aryl, substituted or unsubstituted aralkyl or a substituted or unsubstituted heteroaralkyl; R₂ is —H, a substituted or unsubstituted alkyl, a substituted or unsubstituted aryl, substituted or unsubstituted aralkyl or a substituted or unsubstituted heteroaralkyl; R₃ is a substituted or unsubstituted alkyl, a substituted or unsubstituted aryl, a substituted or unsubstituted heteroaralkyl, or a substituted or unsubstituted heterocycloaralkyl; and R₄ is unsubstituted alkyl, a substituted or unsubstituted aryl, substituted or unsubstituted aralkyl, a substituted or unsubstituted cycloalkylalkyl, a substituted or unsubstituted heteroaryl, a substituted or unsubstituted heteroaralkyl, or a substituted or unsubstituted benzophenonyl; or an active derivative thereof.

In one embodiment, one of R1 and R5 is H and the other is a substituted or unsubstituted phenyl, dicycloalky-C1-C4-alkyl, branched C1-C12-alkyl, cyclic C3-C12-alkyl, phenyl-C1-C4-alkyl. Suitable identities for R1 or R5 include, but are not limited to, 1,2-diphenylethyl, 2-(4-ethylphenyl)ethyl, benzyl, 2,2-diphenylethyl, 2-cyclopentyl-2-phenylethyl, 2-phenyl-2-piridylethyl, diphenylmethyl, 3,3-diphenylpropyl, 3,4,5-tri-methoxybenzyl, 2,4,4-trimethylisopentyl, and 2-(4-methoxyphenyl)ethyl. In other or further embodiments, R2 is a substituted or unsubstituted phenyl, heteroaryl-C1-C4-alkyl, phenylfuranyl, diphenyl-C1-C4-alkyl, and phenyl-C1-C4-alkyl. Suitable identities for R2 include, but are not limited to, phenyl, 2-cyanophenyl, 3-cyanophenyl, 4-cyanophenyl, diphenylmethyl, pyrazolylmethyl, 2,4-dimethylphenyl, 2-methylphenyl, 3-methylphenyl, 4-methylphenyl, 2-methyl-4-methoxyphenyl, 3-methyl-4-methoxyphenyl, 4-methylthiophenyl, 3-chlorophenyl, 3-triflouromethylphenyl, benzyl, 2-trifluoromethylbenzyl, 3-trifluoromethylbenzyl, 2-chlorobenzyl, 3-chlorobenzyl, 4-chlorobenzyl, 2-methoxybenzyl, 3-methoxybenzyl, 4-methoxybenzyl, 2-flurobenzyl, 3-fluorobenzyl, 4-fluorobenzyl, 3-azidylphenyl, and 5-phenylfurna-2-yl. In other or further embodiments, R3 is an alkyl substituted with a heterocylcoalkyl group. Suitable identities for R3 include, but are not limited to, 2-(imidazol-4-yl)ethyl, 3-(imidazol-4-yl)propyl, 3-(imidazol-1-yl)propyl, 2-(3-methylimidazol-4-yl)ethyl, 2-(morpholine-4-yl)ethyl, 2-(4-pyrazol)ethyl, 4-pyrazolylmethyl, 2-N,N-dimethylaminoethyl, 3-N,N-dimethylaminopropyl, and 2-(aminocarbonyl)phenyl. In other or further embodiments, R4 is a substituted or unsubstituted phenyl. Suitable identities for R4 include, but are not limited to, benzophenon-2-yl, 4′-methoxybenzophenon-2-yl, 4′-chlorobenzophenon-2-yl, 2-(furan-2-yl)phenyl, 2-(thiophen-2-yl)phenyl, 2-benzylphenyl, 2-pyridylcarbonylphenyl, 2-(phenoxymethyl)phenyl, 2-(t-butylcarbonyl)phenyl, 2,2-diphenylethyl, 1-fluorenyl, (naphtha-2-yl)methyl, naphtha-1-yl, 3-(phenylcarbonyl)propyl, 4-phenylbutyl, 2-(4-chlorophenylcarbonyl)phenyl, 4-butylphenyl, 2-(4-chlorophenylcarbonyl)phenyl, 3-methoxyphenyl, N-methylpyrrol-2-yl, 2,3-dimethoxyphenyl, 3-butyl-2-pyridyl, 2-naphthylmethyl, 2-cyclohexylethyl, N-methyl-2-pyrrolyl, 2-cyclopentylethyl, 3-oxobutyl, 2-benzopyrazyl, quinoxalin-2-yl, 3-idolyl, (2-methylindol-3-yl)methyl, 3-(indol-3-yl)propyl, (indol-3-yl)methyl, (5-bromoindol-3-yl)-1-hydroxyethyl, 3-fluorophenyl, 1-phenyl-1-hydroxymethyl, 2-phenylphenyl, 2-phenoxyphenyl, thiophen-2-yl, and isopropyl.

In another embodiment, the compound is a ubiquitin-proteasome modulation sensitizing compound. In this embodiment, the compound is present in the composition in an amount effective to potentiate the proteasome inhibitor.

In another embodiment, the compound, designated herein as AS-7, has the molecular formula C₄₂H₃₅N₅O₃ and the following chemical structure:

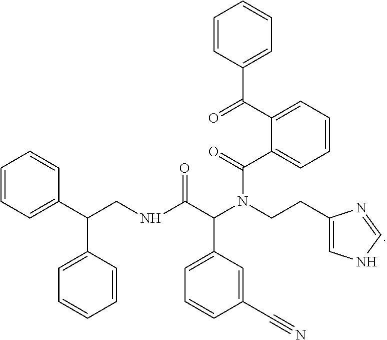 [SMILES: N#Cc1cccc(C(C(=O)NCC(c2ccccc2)c2ccccc2)N(CCc2c[nH]cn2)C(=O)c2ccccc2C(=O)c2ccccc2)c1]

The ubiquitin-proteasome inhibitor can be a proteasome inhibitor or an inhibitor of another component of the ubiquitin-proteasome pathway such as an E3 ubiquitin ligase. Ubiquitin-proteasome inhibitors include, but are not limited to, bortezomib, carfilzomib, disulfiram, epigallocatechin-3-gallate, salinosporamide, lactacystin, clasto-beta-lactone, epoxomicin, eponemycin, dihydroeponemycin, MLN 519, MLN 9708, ONX 0912, CEP-1877, NPI-0052, NLVS (4-hydroxy-5-iodo-3-nitrophenylacetyl-Leu-Leu-leucinal-vinyl sulfone). In one embodiment, the ubiquitin-proteasome inhibitor is bortezomib. In another embodiment, the ubiquitin-proteasome inhibitor is NLVS. Examples of E3 ubiquitin ligase inhibitors include, but are not limited to, MLN 4924, JNJ-26854165, and RG7112.

In accordance with one of the methods, the compound is administered to a cell or subject and a therapeutically effective amount of a ubiquitin-protease inhibitor is also administered to the cell or subject. The compound is administered concurrently with, after, before or between cycles of administration of the proteasome inhibitor. Use of the method results in the potentiation of the ubiquitin-protease inhibitor, and thereby, improves the efficacy of the ubiquitin-proteasome inhibitor. Accordingly, the method improves the efficacy of treatments for cancer, rheumatoid arthritis, autoimmune disease, transplant rejection, multiple sclerosis, sepsis, inflammatory bowel disease, lupus, proliferative disease, benign tumors, hyperplasias, and cachexia. The method is particularly useful for treatment of the multiple myeloma.

In accordance with another one of the methods, an effective amount of the compound is administered to a cell or subject to inhibit cell proliferation.

In accordance with yet another one of the methods an effective amount of the compound is administered to a cell or subject to increase, or augment, glucose utilization.

As used herein, the following terms have the meanings ascribed to them unless specified otherwise.

The term “active derivative” and the like means a modified compound that retains an ability to potentiate a ubiquitin-proteasome inhibitor. Assays for testing the ability of an active derivative compound for potentiating a ubiquitin-proteasome inhibitor are provided in the examples and known to those of skill in the art generally.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “alkyl” refers to straight or branched chain, saturated or mono- or polyunsaturated hydrocarbon groups having from one to 20 carbon atoms and C₁₋ₓ alkyl means straight or branched chain alkyl groups containing from one up to X carbon atoms. For example, C₁₋₆ alkyl means straight or branched chain alkyl groups containing from one up to 6 carbon atoms. Alkoxy means an alkyl-O-group in which the alkyl group is as previously described. Cycloalkyl includes nonaromatic monocyclic or multicyclic ring system, including fused, bridged and spiro rings, of from about three to about 10 carbon atoms. In certain embodiments, cycloalkenyl groups contain four to seven carbon atoms and cycloalkynyl groups contain eight to 10 carbon atoms. A cyclic alkyl may optionally be partially saturated. Cycloalkoxy means a cycloalkyl-O-group in which cycloalkyl is as defined herein.

As used herein, alkyl, alkenyl and alkynyl carbon chains, if not specified, contain from one to 20 carbons, or one to 16 carbons, and are straight or branched. Alkenyl carbon chains from two to 20 carbons, in certain embodiments, contain one to eight double bonds, and the alkenyl carbon chains of two to 16 carbons, in certain embodiments, contain one to five double bonds. Alkynyl carbon chains from two to 20 carbons, in certain embodiments, contain one to eight triple bonds, and the alkynyl carbon chains of two to 16 carbons, in certain embodiments, contain one to five triple bonds. Exemplary alkyl, alkenyl and alkynyl groups include, but are not limited to, methyl, ethyl, propyl, isopropyl, isobutyl, n-butyl, sec-butyl, tort-butyl, isopentyl, tert-pentyl, isohexyl, ethane, propene, butene, pentene, acetylene, and hexyne. As used herein, lower alkyl, lower alkenyl, and lower alkynyl refer to carbon chains having from about one to about two carbons up to about six carbons.

The term “aryl” means an aromatic monocyclic or multicyclic carbocyclic ring system, including fused and spiro rings, containing from about six to 19 carbon atoms. Aryl groups include, but are not limited to, groups such as fluorenyl, substituted fluorenyl, phenyl, substituted phenyl, napthyl and substituted napthyl.

The term “heteroaryl” refers to a monocyclic or multicyclic aromatic ring system, in certain embodiments, of about five to about 15 members where one or more, in one embodiment, one to three, of the atoms in the ring system is a heteroatom (an element other than carbon) including, but not limited to, nitrogen, oxygen and sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

The term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. Specific examples of pharmaceutically acceptable carriers and excipients are provided below.

The term “pharmaceutically acceptable salts” refers to any acid or base addition salt whose counter-ions are non-toxic to the subject to which they are administered in pharmaceutical doses of the salts. Specific examples of pharmaceutically acceptable salts are provided below.

The terms “pharmaceutically effective amount”, “therapeutically effective amount” or “therapeutically effective dose” refer to the amount of a compound such as a ubiquitin-proteasome pathway inhibitor or a ubiquitin-proteasome modulation sensitizing compound that will elicit the biological or medical response of a tissue, system, animal, or human that is being sought by the researcher, veterinarian, medical doctor or other clinician. The term “therapeutically effective amount” includes that amount of a compound such as a ubiquitin-proteasome pathway inhibitor or a ubiquitin-proteasome modulation sensitizing compound that, when administered alone or together, is sufficient to prevent development of, or alleviate to some extent, one or more of the symptoms of the condition or disorder being treated. The therapeutically effective amount will vary depending on the compound such as the ubiquitin-proteasome pathway inhibitor or the ubiquitin-proteasome modulation sensitizing compound, the disorder or conditions and its severity, the route of administration, time of administration, rate of excretion, drug combination, judgment of the treating physician, dosage form, and the age, weight, general health, sex and/or diet of the subject to be treated. In the context of the present method, a pharmaceutically or therapeutically effective amount or dose of a ubiquitin-proteasome inhibitor includes an amount that is sufficient to treat a disease such as cancer, rheumatoid arthritis, autoimmune disease, transplant rejection, multiple sclerosis, sepsis, inflammatory bowel disease, lupus, proliferative disease, benign tumors, hyperplasias, and cachexia.

The terms “potentiate”, “potentiating”, “potentiation” and grammatical variations thereof as used herein, refer to increasing a therapeutic efficacy of a compound. In the context of the present method, the compound is a ubiquitin-proteasome inhibitor. An increase in the therapeutic efficacy of a ubiquitin-proteasome inhibitor can be measured by a decrease in the dosage required to achieve a particular therapeutic result, a decrease in the amount of time or number of treatments required to achieve a particular therapeutic result, or an increase in the scope or type of therapeutic results that can be obtained by administration of a ubiquitin-proteasome inhibitor. It is to be understood that potentiation of a ubiquitin-proteasome inhibitor does not require inhibition of a proteasome by the potentiating compound itself.

The term “potentiation effective amount” includes that amount of a compound, such as a ubiquitin-proteasome modulation sensitizing compound that, when administered to a tissue, system, or subject, is sufficient to potentiate a therapeutic efficacy of another compound, such as a ubiquitin-proteasome inhibitor, by an amount being sought by a researcher, veterinarian, medical doctor or other clinician. The potentiation effective amount will vary depending on the ubiquitin-proteasome modulation sensitizing compound, the ubiquitin-proteasome inhibitor compound, the disorder or condition to be treated and its severity, the route of administration, time of administration, rate of excretion, drug combination, judgment of the treating physician, dosage form, and the age, weight, general health, sex and/or diet of the subject to be treated.

The terms “prevent”, “preventing”, “prevention” and grammatical variations thereof as used herein, refer to a method of partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

The term “proteasome inhibitor” refers to a compound that inhibits one or more activities or functions of a proteasome. Examples of proteasome inhibitors include, but are not limited to, bortezomib, carfilzomib, disulfiram, epigallocatechin-3-gallate, salinosporamide, MLN 9708, ONX 0912, CEP-1877, NPI-0052, and NLVS, a synthetic active center directed, irreversible proteasome inhibitor (4-hydroxy-5-iodo-3-nitrophenylacetyl-Leu-Leu-leucinal-vinyl sulfone), lactacystin, clasto-beta-lactone, epoxomicin, eponemycin, dihydroeponemycin, MLN 519.

The term “subject” is defined herein to include animals such as mammals, including, but not limited to, primates (e.g., humans), cows, sheep, goats, horses, dogs, cats, rabbits, rats, mice and the like. In preferred embodiments, the subject is a human. The term “subject” also includes animal and human patients.

The terms “therapeutic effect” and “therapeutic efficacy” refer to a biological or medical response of a tissue, system, animal, or human that is being sought by a researcher, veterinarian, medical doctor or other clinician and include the treatment of a disease, disorder, or condition.

The terms “treat”, “treating”, “treatment” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively or remedially. In some instances, the terms “treat”, “treating”, “treatment” and grammatical variations thereof, include partially or completely reducing the size of a tumor or cancerous lesion, reducing the number of tumors or cancerous lesions, and reducing the severity of a tumor or cancerous lesion as compared with prior to treatment of the subject or as compared with the incidence of such symptom in a general or study population.

The terms “ubiquitin-proteasome modulation sensitizing compound” and “ubiquitin-proteasome sensitizer” are interchangeable and refer to a compound that sensitizes a cell to the inhibition of one or more activities or functions of a constituent member of the ubiquitin-proteasome pathway other than a TNF-alpha specific pathway member. A TNF-alpha specific pathway member is defined herein as a compound that is required for and restricted to TNF-alpha signaling such as the TNF-alpha receptor. Members of the ubiquitin-proteasome pathway include, but are not limited to, proteasome protein complexes, ubiquitin polypeptides, E1 enzymes, E2 enzymes, and E3 ubiquitin ligases. Accordingly, ubiquitin-proteasome inhibitors include proteasome inhibitors, and E3 ubiquitin ligase inhibitors such as Nedd8-activating enzyme inhibitors and hDM2 inhibitors.

The compound may exist in different isomeric forms. These forms include constitutional, or structural, isomers and stereoisomers. While structural isomers have different bond connections and/or a different order between different atoms or groups, stereoisomers differ in their spatial orientation. Stereoisomers include enantiomers and diastereomers. Diastereomers are further subdivided into cis-trans isomers, conformers, and rotamers. The compound of Formula I, and AS-7 specifically, may also exist in different tautomeric, zwitterionic, and/or stable conformational forms. Spectrographic analysis and other methods known to those of skill in the art can be used to identify the different forms of AS-7 or any other compound of Formula I. Compositions containing the compound of Formula I, or AS-7 specifically, can contain a mixture of different isomeric forms or can contain only one isomeric form.

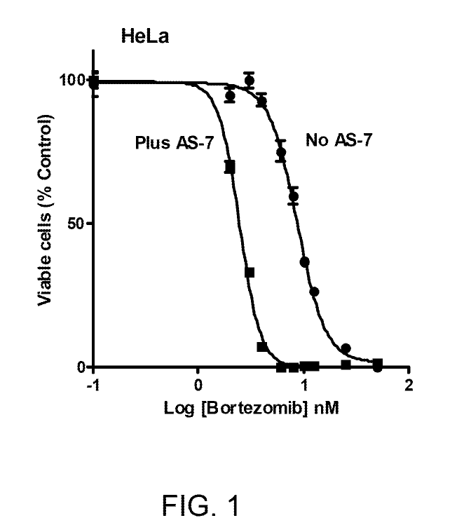

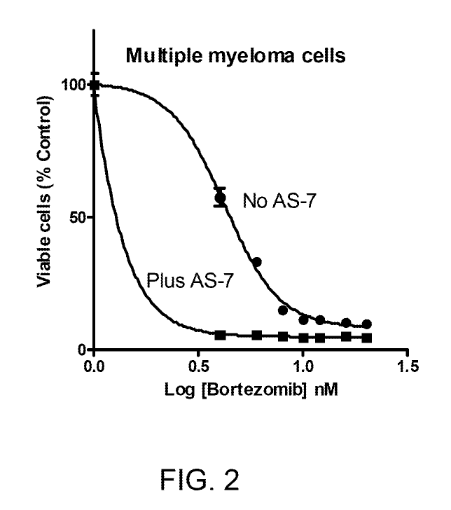

The composition, including the compound and one or more ubiquitin-protease inhibitors, is useful for potentiating the therapeutic efficacy of the ubiquitin-proteasome inhibitor and/or for treating a disease. The examples below demonstrate the surprising finding that ubiquitin-proteasome sensitizer compounds such as AS-7 markedly potentiate the anticancer action of proteasome inhibitors by a mechanism that does not depend on the modulation of tumor necrosis factor (TNF) alpha signaling. While treatment of HeLa and multiple myeloma cells with either bortezomib or structurally distinct NLVS resulted in loss of cell viability, treatment of these same cells with a combination of bortezomib or NLVS and AS-7 resulted in a marked increase in cell death. See FIGS. 1-3. The increased potency of the cytotoxic action of bortezomib was approximately 3.5 fold (FIG. 2) upon the addition of AS-7.

This data demonstrates the synergistic effect that AS-7 has on different protease inhibitors. The terms “synergy”, “synergistic”, “synergism” and the like refer to the production of a greater than expected additive effect of two or more drugs when used in combination. Mathematical analysis of the cytotoxicity data provided above and below in the Examples was performed with CalcuSyn (BioSoft), which confirmed that the combination of AS-7 with a proteasome inhibitor was highly synergistic. CalcuSyn estimates the Dose Reduction Index (DRI), which is a measure of how much the dose of, for example, bortezomib can be reduced at a given effect level relative to the dose of bortezomib alone. At 12 nM bortezomib, analysis of the data for HeLa cells by CalcuSyn indicates a DRI of 7.3 for the combination of AS-7 with bortezomib. The DRI increased to 18 and 72 at 16 and 20 nM, respectively.

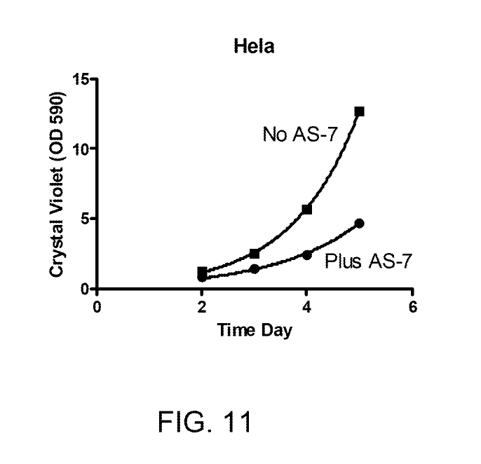

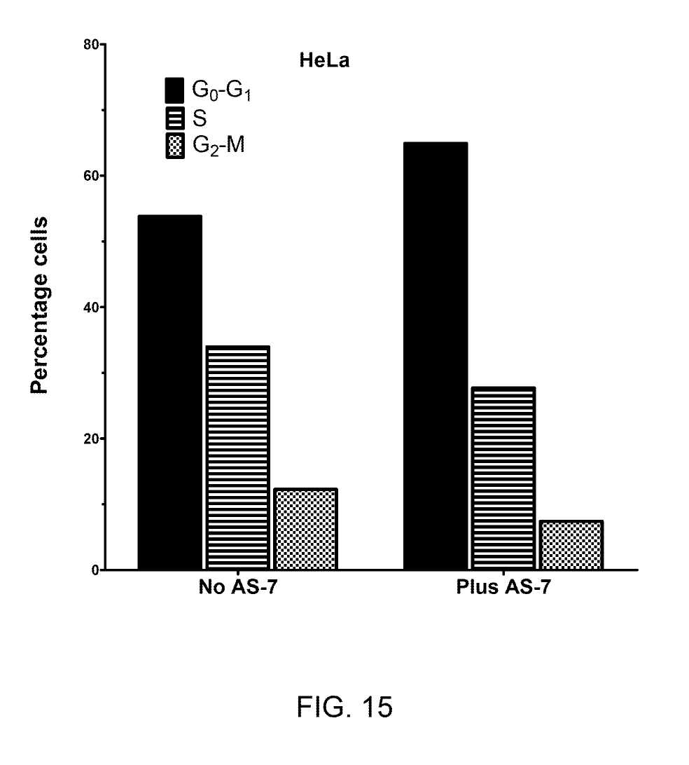

The potentiating or synergistic effect of AS-7 is caused independently of proteasome inhibition by a proteasome inhibiting compound. Treatment of cells with 10 ILIM AS-7 alone for various times had no effect on proteasome activity. AS-7 also did not increase proteasome inhibition by bortezomib (data not shown). However, treatment of HeLa cells with AS-7 alone decreased the rate of growth of the cells (FIG. 11). Together, this data provides evidence that AS-7 sensitizes cells to modulation of the ubiquitin-proteasome pathway via a mechanism that does not directly affect proteasome function. Further data shown in FIG. 15 demonstrates that AS-7 sensitizes cells to modulation of the ubiquitin-proteasome pathway via a mechanism that is independent of the modulation of TNF-alpha specific signaling.

As mentioned above, a method is provided that includes administering to a cell or subject one or more ubiquitin-proteasome inhibitors and a potentiation effective amount of a ubiquitin-proteasome sensitizer compound, wherein the compound potentiates a therapeutic effect of the one or more ubiquitin-proteasome inhibitors. Also provided is a method of treating a disease in a subject by administering to the subject a therapeutically effective amount of one or more proteasome inhibitors and a ubiquitin-proteasome sensitizer compound.

The compound used in the methods has the chemical formula set forth above. In some embodiments, the method includes administering to a subject the ubiquitin-proteasome inhibitor and a potentiation effective amount of a ubiquitin-proteasome sensitizer compound having the chemical formula of AS-7 as described above, wherein the sensitizer compound potentiates a therapeutic effect of the proteasome inhibitor. In another embodiment, the method includes administering to a cell or subject a therapeutically effective amount of a proteasome inhibitor and a ubiquitin-proteasome sensitizer compound for the treatment of a disease.

The ubiquitin-proteasome inhibitors used in the method include proteasome inhibitors. In some embodiments, the proteasome inhibitor is selected from the group consisting of bortezomib, carfilzomib, MLN 9708, ONX 0912, CEP-1877, NPI-0052, and NLVS. In one embodiment, the proteasome inhibitor is bortezomib. In another embodiment, the proteasome inhibitor is NLVS. Ubiquitin-proteasome inhibitors also include inhibitors of other components of the ubiquitin-proteasome pathway such as an E3 ubiquitin ligase. Accordingly, the method includes the use of E3 ubiquitin ligase inhibitors such as MLN 4924, JNJ-26854165, and RG7112.

The therapeutic effect that is potentiated using the method can be any therapeutic effect associated with the administration of a ubiquitin-proteasome inhibitor. Therapeutic effects and/or diseases associated with the method include, but are not limited to, treatment of proliferative diseases, including but not limited to cancer, rheumatoid arthritis, autoimmune disease, transplant rejection, multiple sclerosis, sepsis, inflammatory bowel disease, lupus, proliferative disease, benign tumors, hyperplasias, and cachexia. In one embodiment, the therapeutic effect is the treatment of a cancer. The cancer can be selected from astrocytoma, adrenocortical carcinoma, appendix cancer, basal cell carcinoma, bile duct cancer, bladder cancer, bone cancer, brain cancer, brain stem glioma, breast cancer, cervical cancer, colon cancer, colorectal cancer, cutaneous T-cell lymphoma, ductal cancer, endometrial cancer, ependymoma, Ewing sarcoma, esophageal cancer, eye cancer, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer and Wilms tumor. In some embodiments, the cancer is multiple myeloma.

The ubiquitin-proteasome sensitizer compounds are administered before, concurrently, or after administration of the proteasome inhibitor. In one embodiment, the ubiquitin-proteasome sensitizer compound is administered concurrently with the proteasome inhibitor. In another embodiment, the ubiquitin-proteasome sensitizer compound is administered before administration of a proteasome inhibitor. When administered before a proteasome inhibitor, the ubiquitin-proteasome sensitizer compound is preferably administered 1, 2, 3, 4, 5, 6, 8, 10, 12, 18, 24, 30, or 45 minutes, hours, or days prior to the proteasome inhibitor administration. In those embodiments that include bortezomib as a proteasome inhibitor, bortezomib can be dosed at 1.3 to 1.5 mg/m² of subject body surface on day 1, 4, 8, 11 and 21 and repeating such regimen after a two week interval.

It will be appreciated that the ubiquitin-proteasome sensitizer compound and/or the ubiquitin-proteasome inhibitor can be administered as a pharmaceutically acceptable salt, ester or pro-drug thereof, or in a pharmaceutically acceptable carrier or diluent. The ubiquitin-proteasome sensitizer compound and/or the ubiquitin-proteasome inhibitor described herein may be derivatized at functional groups to provide pro-drug derivatives that are capable of conversion back to the parent compounds in vivo. Examples of such pro-drugs include the physiologically acceptable and metabolically labile ester derivatives, such as methoxymethyl esters, methoyltiomethyl esters, or pivaloyloxymethyl esters derived from a hydroxyl group of a ubiquitin-proteasome sensitizer compound and/or a ubiquitin-proteasome inhibitor or a carbamoyl moiety derived from an amino group of a ubiquitin-proteasome sensitizer compound and/or a ubiquitin-proteasome inhibitor. Additionally, any physiologically acceptable equivalents of the ubiquitin-proteasome sensitizer compound and/or the ubiquitin-proteasome inhibitor, similar to metabolically labile esters or carbamates, that are capable of producing the ubiquitin-proteasome sensitizer compound and/or the ubiquitin-proteasome inhibitor in vivo, are within the scope of the method provided herein.

If pharmaceutically acceptable salts of the ubiquitin-proteasome sensitizer compound and/or the ubiquitin-proteasome inhibitor are utilized for administration, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexamoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, priopionate, succinate, tartrate, thiocynate, tosylate, undecanoate, hydrohalides, sulfates, phosphates, nitrates, sulphamates, malonates, salicylates, methylene-bix-b-hydroxynaphthoates, gentisates, isethionates, di-p-toluoyltartrates, ethanesulphonates, cyclohexylsulphamates, quinates, and the like. Pharmaceutically base addition salts include, but are not limited to, those derived from alkali or alkaline earth metal bases or conventional organic bases, such as tri-ethylamine, pyridine, piperidine, morpholine, N-methylmorpholine, ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, etc. Furthermore, the basic nitrogen-containing groups may be quaternized with agents such as lower alkyl halides, such as methyl, ethyl, propyl and butyl chlorides, bromides and iodides, dialkyl sulfates, such as dimethyl, diethyl, dibutyl and diamyl sulfates, long chain halides, such as decyl, lauryl, myristyl and stearyl chlorides, bromides and iodides, aralkyl halides, such as benzyl and phenethyl bromides and others. Water or oil-soluble or dispersible products are thereby obtained.

The ubiquitin-proteasome sensitizer compound and/or the ubiquitin-proteasome inhibitor compounds utilized in the method provided herein may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion. In particular, the ubiquitin-proteasome sensitizer compound such as AS-7 is a hydrophobic compound with limited solubility in aqueous solutions. In some embodiments, AS-7 or an active derivative thereof can be dissolved at 50 mM AS-7 in dimethylsulfoxide containing 5% Cremaphor® EL detergent (Sigma Aldrich) and diluted approximately one thousand fold in a physiologic salt solution such as saline containing albumin in the range of the albumin concentration of blood. Alternatively, nanoparticles containing AS-7 or an active derivative thereof are administered to the subject. Nanoparticles can be made by mixing human albumin at a 3-4% solution in a salt solution of the desired pH and ionic strength, subjecting the solution to elevated pressure and releasing the solution via a small jet. Nanoparticles of optimal size can be purified by differential centrifugation. These methods are known by those of skill in the art and can be found in further detail in Katz, F., J. of Controlled Release 132:171-183, 2008.

The ubiquitin-proteasome sensitizer compound and/or the ubiquitin-proteasome inhibitor can be manufactured by methods well known in the art such as conventional granulating, mixing, dissolving, encapsulating, lyophilizing, or emulsifying processes, among others. Compositions containing the ubiquitin-proteasome sensitizer compound and/or the ubiquitin-proteasome inhibitors may be produced in various forms including granules, precipitates, particulates, powders, tablets, capsules, syrup, suppositories, injections, emulsions, elixirs, suspensions or solutions. These compositions may optionally contain stabilizers, pH modifiers, surfactants, bioavailability modifiers and combinations of these.

Pharmaceutically acceptable carriers that may be administered with the ubiquitin-proteasome sensitizer compound and/or the ubiquitin-proteasome inhibitor include ion exchangers, alumina, aluminum stearate, lecithin, serum proteins, such as human serum albumin, buffer substances, such as phosphates, glycine, sorbic acid, potassium sorbate, partial glyceride mixtures of vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The ubiquitin-proteasome sensitizer compound and/or the ubiquitin-proteasome inhibitor may additionally be administered in compositions that include lubricating agents such as talc, magnesium stearate, and mineral oil; wetting agents; emulsifying agents; suspending agents; preserving agents such as methyl-, ethyl-, and propyl-hydroxy-benzoates; pH adjusting agents such as inorganic and organic acids and bases; sweetening agents; and flavoring agents.

Administration of a ubiquitin-proteasome sensitizer compound and/or the ubiquitin-proteasome inhibitor with one or more suitable pharmaceutical excipients as advantageous are carried out via any of the accepted modes of administration and as the term “administering” is defined above. Preferably, the ubiquitin-proteasome sensitizer compound and/or the ubiquitin-proteasome inhibitor are administered orally. The ubiquitin-proteasome sensitizer compound and/or the ubiquitin-proteasome inhibitor can be administered once or repeatedly, e.g. at least 2, 3, 4, 5, 6, 7, 8, or more times, or by continuous infusion.

### EXAMPLES

The method provided herein will be further understood by reference to the following non-limiting examples.

### Example I

AS-7 Potentiates the Cytotoxic Action of Anticancer Drug Bortezomib in HeLa Cells

HeLa cells were seeded in a 96 well tissue culture plate (2000 cells per well). The compounds, 10 μM AS-7 and bortezomib (BioVision, Inc., Milpitas, Calif.) at the concentrations indicated in FIG. 1, were added the following day. AS-7 was prepared according to the Ugi reaction by combination of the four components: isocyanide (III), m-cyanobenzaldehyde (IV), 2-benzoylbenzoic acid (V), and histamine (VI) in refluxing methanol (Ref. http://www.occhem.com/synthesis/2561 2.html). The Medicinal Chemistry unit of Southern Research Institute (2000 Ninth Avenue South Birmingham, Ala. 35205) was contracted by a fee for service agreement to synthesize AS-7.

After 48 hours, MTS (3-(4,5-dimethylthiazol-2-yl)-5-(3-carboxymethoxyphenyl)-2-(4-sulfophenyl)-2H-tetrazolium) was added and absorbance was measured at 490 nm. The data are shown in FIG. 1. These data are mean±SD, n=3. Values in FIG. 1 are relative to controls that contained only the vehicle in which the drugs were dissolved. Vehicle had no effect on cell viability. Error bars in FIG. 1 are not visible if they are smaller than the symbols.

FIG. 1 demonstrates that AS-7 markedly potentiates the cytotoxic action of bortezomib as indicated by the loss of HeLa cell viability. FIG. 1 further shows that AS-7 markedly shifts the concentration dependence of bortezomib to the left, i.e., to lower concentrations. The IC50 (the 50% inhibitory concentration) of bortezomib was approximately 8.8 nM in the absence of AS-7 and 2.5 nM in the presence of AS-7. The IC50 values were determined by nonlinear regression analysis of the data in FIG. 1. Thus AS-7 increased the potency of the cytotoxic action of bortezomib by approximately 3.5 fold.

Importantly, treatment of HeLa cells with AS-7 alone moderately decreased the rate of growth of HeLa cells, but produced no loss of cell viability. Therefore, the combination of AS-7 with a proteasome inhibitor (bortezomib or NLVS) produces a synergistic loss of cell viability. AS-7 produced the synergistic loss of cell viability in the concentration range of 2 to 10 μM (data not shown).

The cytotoxic action of bortezomib is known to be mediated by proteasome inhibition and the induction of apoptosis. Proteasome activity was measured in cell lysates and shown to be markedly inhibited in the bortezomib treated HeLa cells (data not shown). Additionally, the treatment of the HeLa cells with bortezomib increased the caspase-3 activity measured in cell lysates, indicating that induction of apoptosis accounted for at least part of the loss of cell viability (data not shown).

### Example II

### AS-7 Potentiates the Cytotoxic Action of Anticancer Drug Bortezomib in Multiple Myeloma Cells

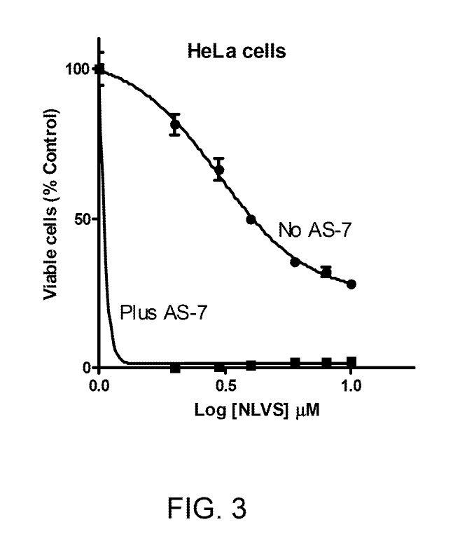

Multiple myeloma cells (RPMI 8226) were seeded in a 96 well tissue culture plate (2000 cells per well). The compounds, 10 μM AS-7 and bortezomib at the concentrations indicated in FIG. 3, were added the following day. AS-7 was prepared as described in Example I above. After 48 hours, MTS was added and absorbance was measured at 490 nm. The data are shown in FIG. 1. These data are mean±SD, n=3. Values in FIG. 2 are relative controls that contained only the vehicle in which the drugs were dissolved. Vehicle had no effect on cell viability. Error bars in FIG. 2 are not visible if they are smaller than the symbols.

FIG. 2 demonstrates that AS-7 markedly potentiates the cytotoxic action of bortezomib as indicated by the loss of multiple myeloma cell viability. FIG. 2 further shows that AS-7 markedly shifts the concentration dependence of bortezomib to the left, i.e., to lower concentrations.

### Example III

### AS-7 Potentiates the Cytotoxic Action of NVLS in HeLa Cells

HeLa cells were seeded in a 96 well tissue culture plate (2000 cells per well). The compounds, 10 μM AS-7 and NLVS at the concentrations indicated in FIG. 3 were added the following day. AS-7 was prepared as described in Example I above. NLVS is a synthetic active center directed, irreversible proteasome inhibitor (4-hydroxy-5-iodo-3-nitrophenylacetyl-Leu-Leu-leucinal-vinyl sulfone) (Bogyo M, McMaster J S, Gaczynska M, Tortorella D, Goldberg A L, Ploegh H. (1997) Proc Natl Acad Sci USA 94:6629-6634).

After 48 hours, MTS was added and absorbance was measured at 490 nm. The data are shown in FIG. 3. These data are mean±SD, n=3. Values in FIG. 3 are relative controls that contained only the vehicle in which the drugs were dissolved. Vehicle had no effect on cell viability. Error bars in FIG. 3 are not visible if they are smaller than the symbols.

FIG. 3 demonstrates that the potentiation of the cytotoxic action of proteasome inhibitors by AS-7 is not limited to bortezomib. AS-7 similarly sensitized HeLa cells to another proteasome inhibitor, NLVS. FIG. 3 shows that AS-7 markedly shifted the NLVS concentration dependence to the left as shown for bortezomib. Concentrations of NLVS that, by themselves, had little effect on cell viability produced a complete loss of viability in the presence of AS-7 (FIG. 3). Therefore, AS-7 appears to synergize with the entire class of drugs that block the proteasome.

### Example IV

### AS-7 Fails to Potentiate the Cytotoxicity of Paclitaxel and Doxorubicin in HeLa Cells

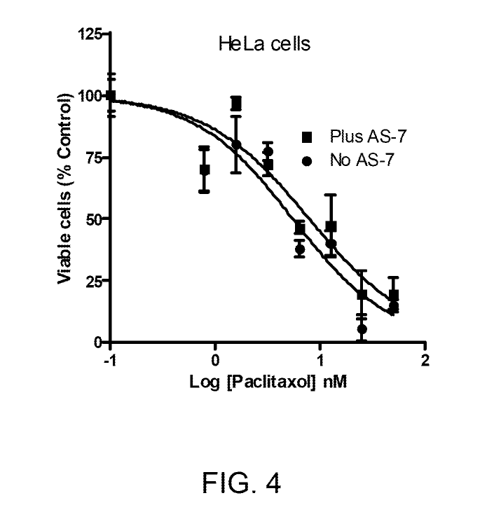

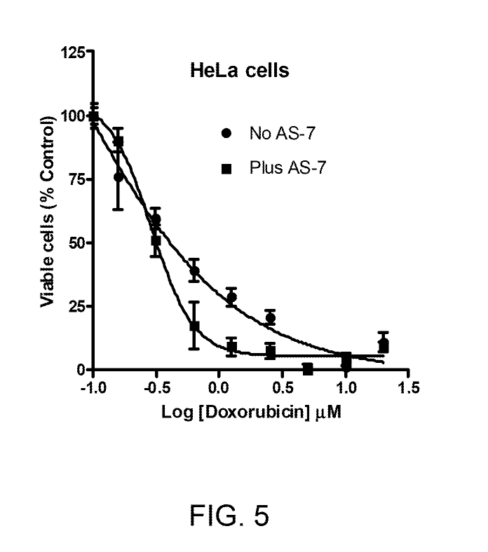

HeLa cells were seeded in a 96 well tissue culture plate (2000 cells per well). The compounds, 10 μM AS-7 and paclitaxel and doxorubicin at the concentrations indicated in FIGS. 4 and 5, respectively, were added the following day. AS-7 was prepared as described in Example I above. After 48 hours, MTS was added and absorbance was measured at 490 nm. The data are shown in FIGS. 4 and 5. These data are mean±SD, n=3. Values in FIGS. 4 and 5 are relative controls that contained only the vehicle in which the drugs were dissolved. Vehicle had no effect on cell viability. Error bars in FIGS. 4 and 5 are not visible if they are smaller than the symbols.

FIG. 4 shows that AS-7 had no effect on the cytotoxicity of paclitaxel. AS-7 has no effect on the paclitaxel concentration that decreased HeLa cell viability by 50% (IC50) (FIG. 4). FIG. 5 further shows that AS-7 has no significant effect on the cytotoxicity of the anticancer drug doxorubicin. AS-7 had no effect on the IC50 of doxorubicin. These data indicate that AS-7 interacts selectively with the proteasome inhibitor class of anticancer drugs because AS-7 failed to generally potentiate the cytotoxic action of two structurally diverse anticancer drugs, paclitaxel and doxorubicin.

### Example V

### AS-7 Potentiates the Cytotoxic Action of Anticancer Drug Bortezomib in Ovarian IGROV and A2780 Cells

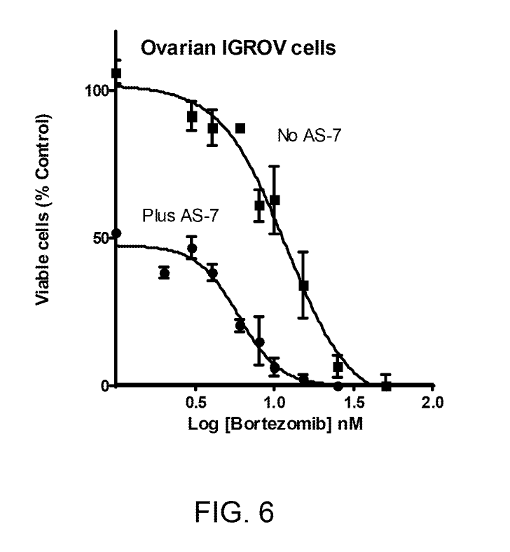

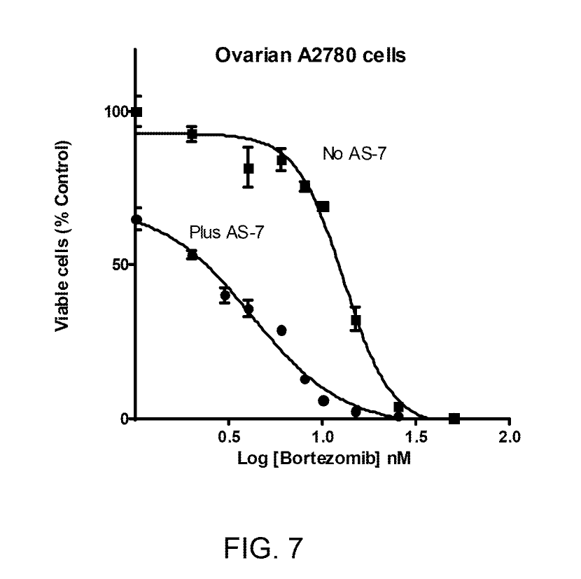

Ovarian tumor-derived cell lines, IGROV and A2780, were seeded in a 96 well tissue culture plate (2000 cells per well). The compounds, 10 μM AS-7 and bortezomib (BioVision, Inc., Milpitas, Calif.) at the concentrations indicated in the figures, were added the following day. AS-7 was prepared as described in Example I above. After 72 hours the cells were fixed with 4% paraformaldehyde (4° C. for 30 minutes), stained for 30 minutes with 0.5% crystal violet in 10% ethanol, and washed extensively with water to remove dye that was not bound to the cells. Cell bound crystal violet was solubilized with 10% acetic acid and absorbance was measured at 590 nm. The data are shown in FIG. 6 and FIG. 7. These data are mean±SD, n=3. Values are relative to cells that with the vehicle in which the drugs were dissolved. Vehicle had no effect on cell viability. Error bars in FIG. 6 and FIG. 7 are not shown if they are smaller than the symbols.

The data on both ovarian tumor cell lines demonstrate that AS-7 markedly potentiates the cytotoxic action of bortezomib as indicated by the loss of cell viability. FIG. 7 further shows that AS-7 markedly shifts the concentration dependence of bortezomib to the left, i.e., to lower concentrations. The IC50 (the 50% inhibitory concentration) of the cytotoxic effect of bortezomib towards the ovarian A2780 cells was approximately 12.9 nM in the absence of AS-7 and 4.2 nM in the presence of AS-7. The IC50 values were determined by nonlinear regression analysis of the data using GraphPad Prism. Thus, AS-7 increased the potency of the cytotoxic action of bortezomib by approximately 3.1 fold.

### Example VI

### AS-7 Potentiates the Cytotoxic Action of Anticancer Drug Bortezomib in Melanoma A2620 Cells

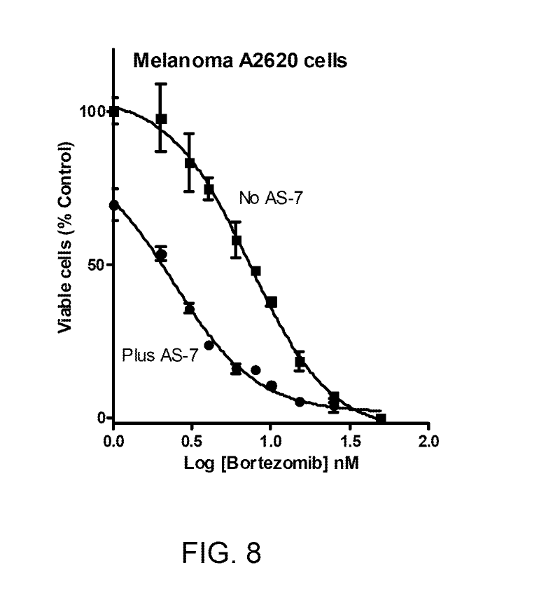

Melanoma A2620 cells were seeded in a 96 well tissue culture plate (2000 cells per well). The compounds, 10 μM AS-7 and bortezomib at the concentrations indicated in FIG. 8 were added the following day. After 72 hours the cells were fixed with 4% paraformaldehyde (4° C. for 30 minutes), stained for 30 minutes with 0.5% crystal violet in 10% ethanol, and washed extensively with water to remove dye that was not bound to the cells. Cell bound crystal violet was solubilized with 10% acetic acid and absorbance was measured at 590 nm. The data are shown in FIG. 8. These data are mean±SD, n=3. Values are relative to controls that contained only the vehicle in which the drugs were dissolved. Vehicle had no effect on cell viability.

FIG. 8 demonstrates that AS-7 markedly potentiates the cytotoxic action of bortezomib as indicated by the loss of HeLa cell viability. FIG. 8 further shows that AS-7 markedly shifts the concentration dependence of bortezomib to the left, i.e., to lower concentrations. The IC50 (the 50% inhibitory concentration) of bortezomib was approximately 7.3 nM in the absence of AS-7 and 2.4 nM in the presence of AS-7. The IC50 values were determined by nonlinear regression analysis. Thus AS-7 increased the potency of the cytotoxic action of bortezomib by approximately three fold.

### Example VII

### AS-7 Potentiates the Cytotoxic Action of Anticancer Carfilzomib in HeLa Cells

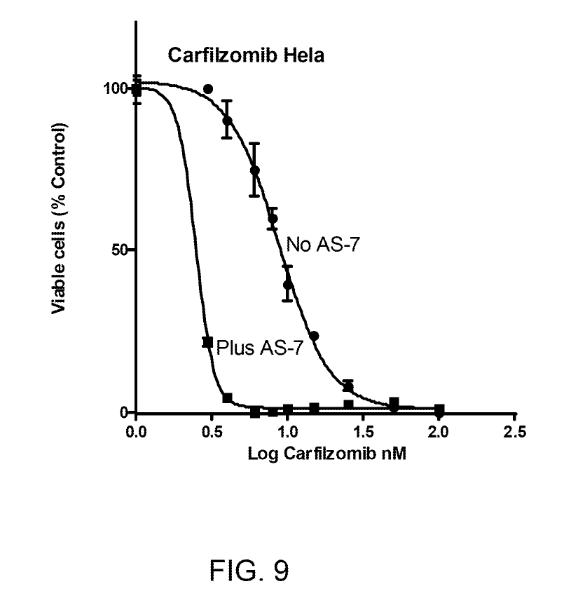

HeLa cells were seeded in a 96 well tissue culture plate (2000 cells per well). The compounds, 10 μM AS-7 and bortezomib at the concentrations indicated in FIG. 9 were added the following day. After 48 hours, MTS was added and absorbance was measured at 490 nm. The data are shown in FIG. 9. These data are mean±SD, n=3. Values are relative to controls that contained only the vehicle in which the drugs were dissolved. Vehicle had no effect on cell viability.

FIG. 9 demonstrates that AS-7 markedly potentiates the cytotoxic action of carfilzomib as indicated by the loss of HeLa cell viability. FIG. 9 further shows that AS-7 markedly shifts the concentration dependence of carfilzomib to the left, i.e., to lower concentrations. The IC50 (the 50% inhibitory concentration) of carfilzomib was approximately 8.5 nM in the absence of AS-7 and 2.5 nM in the presence of AS-7. The IC50 values were determined by nonlinear regression analysis. Thus AS-7 increased the potency of the cytotoxic action of carfilzomib by approximately 3.5 fold.

### Example VIII

### AS-7 Potentiates the Cytotoxic Action of Anticancer Drug Bortezomib in Breast Cancer Cells

Breast cancer derived cell lines, T47D and 159C, were seeded in a 96 well tissue culture plate (2000 cells per well). The significance of these two lines of breast cancer derived cell lines is that the T47D cells positive for three markers used to optimize the chemotherapeutic treatment, namely HER2 receptor, estrogen receptor, and progesterone receptor. By contrast to the T47D cells, the 159C cells are negative for the three markers. The compounds, 10 μM AS-7 and a range of bortezomib (BioVision, Inc., Milpitas, Calif.) concentrations were added the following day. After 48 hours, MTS was added and absorbance was measured at 490 nm. Both breast cancer cell lines were highly sensitive to the cytotoxic action of bortezomib by itself. Moreover, AS-7 markedly potentiated the cytotoxic action of bortezomib as indicated by the loss of cell viability and decrease in the IC50 values of bortezomib in the presence of AS-7 compared to the IC50 values of bortezomib by itself (data not shown).

### Example IX

### AS-7 by Itself Inhibits the Proliferation Human Cancer Cell Lines

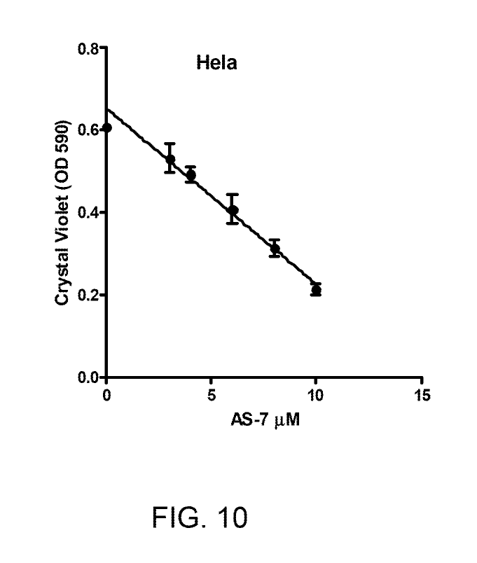

Cell lines were plated at a sparse density in order to measure the rate of growth over a several day interval and obviate the effect of limited culture surface on the growth rate. The impact of AS-7 on cell proliferation became clear after two days and was dramatic after several days. While the impact of AS-7 was modest after two days, it became substantial after more than two days because the reduction in growth rate has the greatest impact on cell number after the cultures have expanded by several population doublings. Titration of AS-7 clearly showed that the concentration dependence of growth inhibition was essentially the same as the AS-7 concentration dependence of the potentiation of the cytotoxicity of bortezomib. The IC50 for growth inhibition by AS-7 was approximately 7 μM (FIG. 10). AS-7 inhibited the rate of cell proliferation by 67% at a concentration of approximately 10 μM (FIG. 10).

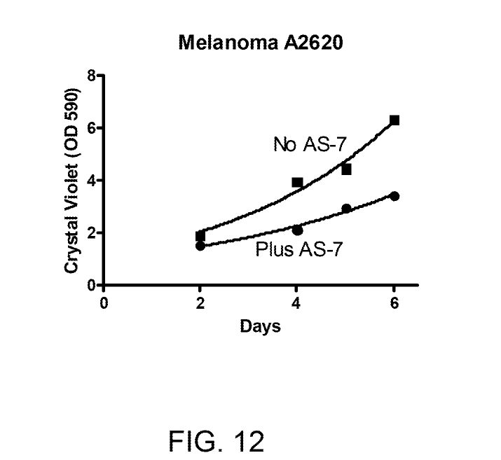

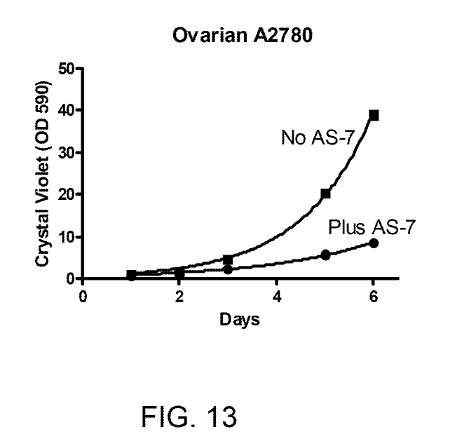

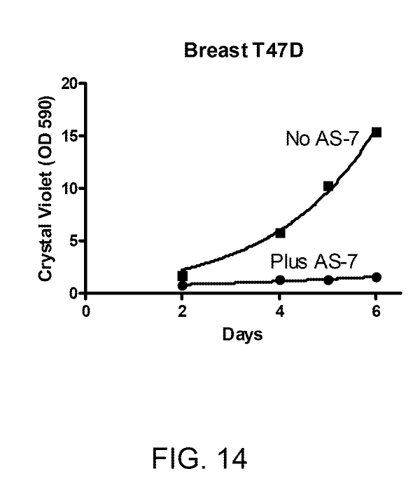

To examine the effect of AS-7 on the growth of several different cell lines, AS-7 at 10 μM was added to the culture medium the day after seeding the cells. The following cell lines were used to determine the effect of AS-7 on the rate of proliferation: HeLa, melanoma A2620, ovarian A2780, and breast T47D. All cell line were grown in culture medium (RPMI 1640 for the breast cancer cells and Dulbecco's modified Eagle's medium for the other cell lines) containing 10% heat inactivated fetal bovine serum without antibiotics (penicillin and streptomycin). After the indicated number of days (FIGS. 11 through 14), the cells were fixed with 4% paraformaldehyde (4° C. for 30 minutes), stained for 30 minutes with 0.5% crystal violet in 10% ethanol, and washed extensively with water to remove dye that was not bound to the cells. Cell bound crystal violet was solubilized with 10% acetic acid and absorbance was measured at 590 nm to quantitate cell growth. The time course data for each cell line were fit by nonlinear regression analysis to the equation for exponential growth using GraphPad Prism software. The data for each cell line treated with vehicle only or 10 μM AS-7 gave excellent fit as shown in FIGS. 11 through 14. The doubling times in HeLa, melanoma, ovarian and breast cancer derived cells were 0.9, 2.5, 1.0, and 1.4 days, respectively. AS-7 increased the doubling times of the HeLa, melanoma, ovarian, and breast cancer derived cells by 1.3, 1.3, 1.6, and 3.1 fold, respectively (FIGS. 11 through 14).

AS-7 substantially inhibited the rate of cell growth of each of the cancer-derived cell lines tested. AS-7 by itself did not decrease cell viability, and no cell death, cell rounding or detachment from the culture surface was detectable by microscopic observation of the cultures. The appearance of essentially all of the AS-7 treated cells was indistinguishable from that of the untreated cells. Additionally treatment of HeLa cells with AS-7 for several days failed to induce apoptosis as indicated by FACS (Fluorescence Activated Cell Sorter) analysis after propidium iodide staining (data not shown).

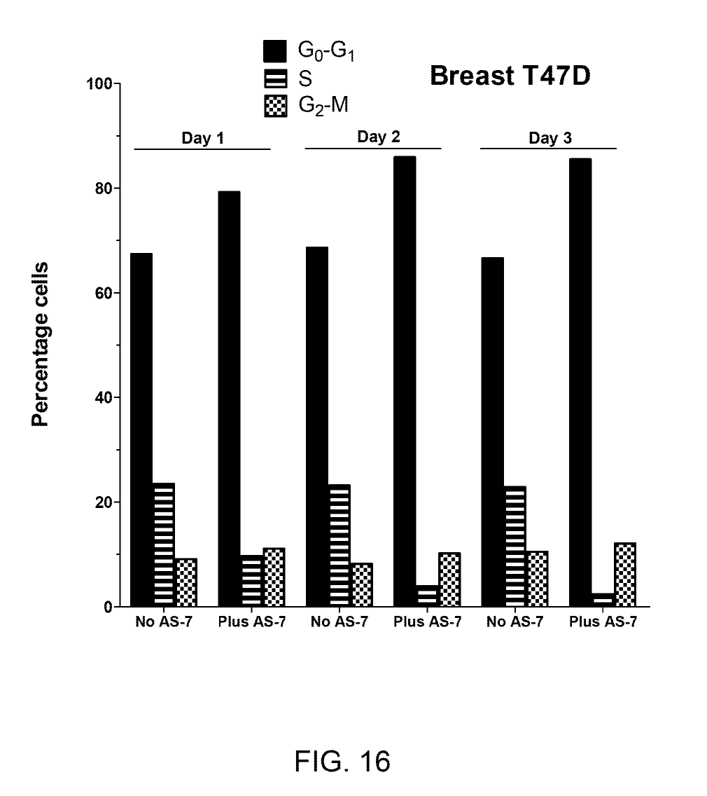

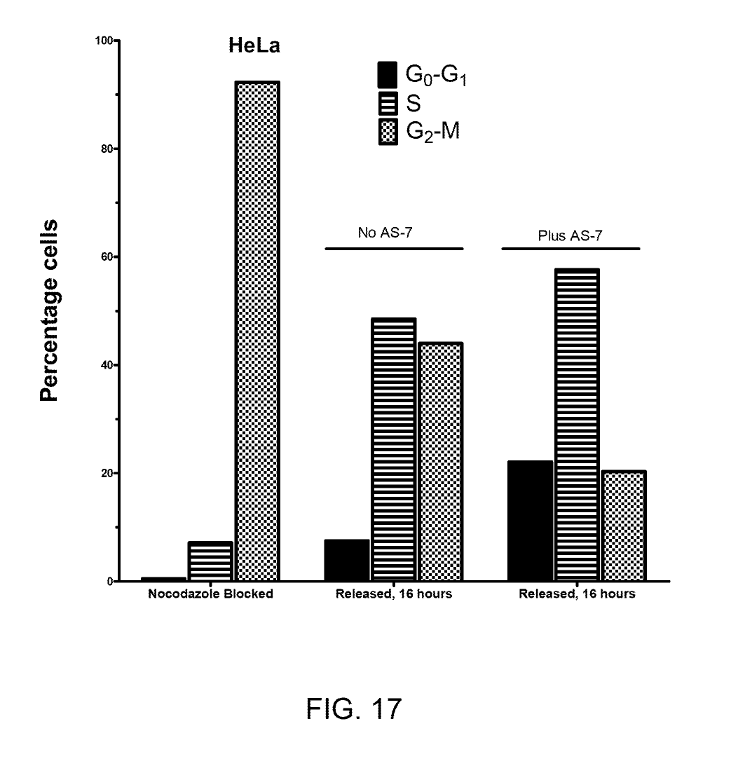

AS-7 (10 μM) evoked the accumulation of HeLa and breast T47D cells in the Gₒ-G₁ phase of the cell cycle (FIGS. 15 and 16) as indicated by FACS (Fluorescence Activated Cell Sorter) analysis after propidium iodide staining. A single addition of 10 μM AS-7 for 1 day to 7 or more days was sufficient to evoke accumulation in the Gₒ-G₁ phase of the cell cycle (FIGS. 15 and 16) (data not shown). FIG. 17 shows that AS-7 blocked exit from the G₀-G₁ phase of the cell cycle. HeLa cells were treated with nocodazole (100 ng/ml) for 12 h which evoked accumulation of rounded, loosely attached cells in mitosis. The mitotic cells were harvested and treated with or without 10 μM AS-7 in the absence of nocodazole (Released, 16 hours). AS-7 evoked the accumulation of the cells in the G₀-G₁ phase of the cell cycle without affecting exit from G₂-M or entry into the S phase of the cell cycle (FIG. 17).

### Example X

### AS-7 Fails to Block the Cytotoxic Action of TNF-alpha

TNF-alpha is well known to induce caspase-mediated apoptosis in a wide variety of cultured mammalian cells, including HeLa cells, when transcription or translation is blocked by the addition of actinomycin D or cycloheximide, respectively, together with TNF-alpha. HeLa cells were treated with 10 micrograms/ml cycloheximide alone or together with a range of TNF-alpha concentrations from 2.5 to 20 nanograms per ml. Following treatment, the cells were fixed with 4% paraformaldehyde (4° C. for 30 minutes), stained for 30 minutes with 0.5% crystal violet in 10% ethanol, and washed extensively with water to remove dye that was not bound to the cells. Cell bound crystal violet was solubilized with 10% acetic acid and absorbance was measured at 590 nm to quantitate cell growth. The time course data for each cell line were fit by nonlinear regression analysis to the equation for exponential growth using GraphPad Prism software.

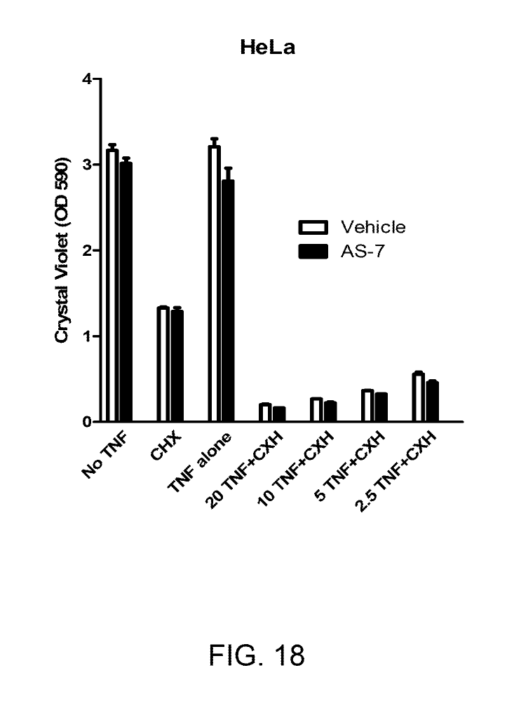

Treatment with cycloheximide inhibited cell proliferation, but failed to induce cell death in either the absence or presence of 10 nM AS-7 (FIG. 18). Thus cycloheximide was cytostatic, but not cytotoxic to the cells over a 24 hour interval. The combination of cycloheximide with TNF-alpha was rapidly cytotoxic which was apparent within six hours of treatment.

Some HeLa cells were pretreated with 10 nM AS-7 for two hours to determine whether or not AS-7 could modulate the cytotoxicity of TNF-alpha as shown for human fibroblasts for Compound 44 as disclosed in U.S. Patent Application Publication No. 2002/011998. Importantly, the pretreatment and continuous presence of AS-7 during the TNF-alpha treatment failed to block the cytotoxicity evoked by TNF-alpha at any of the concentrations of TNF-alpha tested (FIG. 18). These data conclusively indicate that the modulation of TNF-alpha action (or modulation of a TNF-alpha specific pathway member) mediates neither the synergistic potentiation of the cytotoxicity of ubiquitin-proteasome pathway inhibitors by AS-7, nor the growth inhibitory effects of AS-7.

### Example XI

### AS-7 by Itself Increases Glucose Utilization and Acid Production

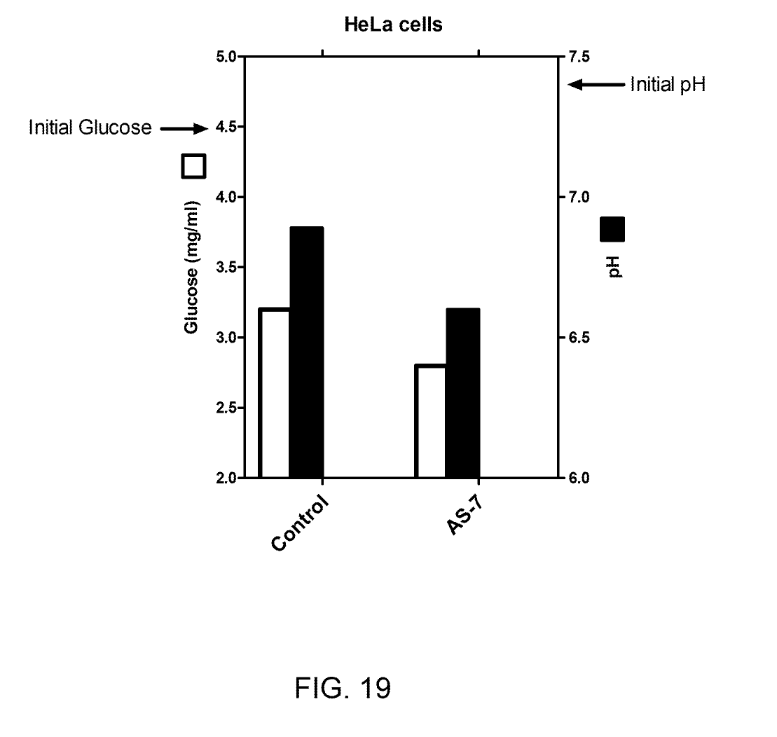

Because it was repeatedly observed that AS-7 by itself decreased the pH of the culture medium, it was hypothesized that AS-7 might stimulate glucose utilization and the production of lactic acid, which is a major acidic product of glucose metabolism. HeLa cells were cultured in 100 millimeter diameter tissue culture plates in order to monitor the pH and the glucose concentration of the culture medium. Glucose was quantified with a glucose test strip and meter, as commonly used to monitor fluctuations in blood glucose. A pH meter was used to record the pH of the medium. Control cultures (“Control”) were treated with the appropriate amount of the vehicle used dissolve AS-7. Treated cultures (“AS-7”) incubated with 10 μM AS-7. After 5 days AS-7 significantly decreased both the glucose concentration and the pH of the culture medium relative to control cultures treated with vehicle alone (FIG. 19). While AS-7 stimulated glucose utilization, the magnitude of the effect is underestimated because the AS-7 treatment inhibited HeLa cell growth as shown above in FIG. 18 and arrested the cells in the G0-G1 phase of the cell cycle (FIG. 15 and FIG. 17).

It is understood that the examples and embodiments described herein are for illustrative purposes only and that various modifications or changes in light thereof will be suggested to person skilled in the art and are to be included with the spirit and purview of this applications and scope of the appended claims. All publications, patents and patent applications cited herein are hereby incorporated by reference in their entirety for all purposes.

What is claimed is:

1. A composition comprising a proteasome inhibitor and a compound having the following chemical formula: 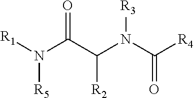
wherein R1 and R5 are each, independently, —H, a substituted or unsubstituted alkyl, a substituted or unsubstituted aryl, substituted or unsubstituted aralkyl or a substituted or unsubstituted heteroaralkyl; R2 is —H, a substituted or unsubstituted alkyl, a substituted or unsubstituted aryl, substituted or unsubstituted aralkyl or a substituted or unsubstituted heteroaralkyl; R3 is a substituted or unsubstituted alkyl, a substituted or unsubstituted aryl, a substituted or unsubstituted heteroaralkyl, or a substituted or unsubstituted heterocycloaralkyl; and R4 is unsubstituted alkyl, a substituted or unsubstituted aryl, substituted or unsubstituted aralkyl, a substituted or unsubstituted cycloalkylalkyl, a substituted or unsubstituted heteroaryl, a substituted or unsubstituted heteroaralkyl, or a substituted or unsubstituted benzophenonyl; or an active derivative thereof.

2. The composition of claim 1 wherein the compound has the following chemical formula:

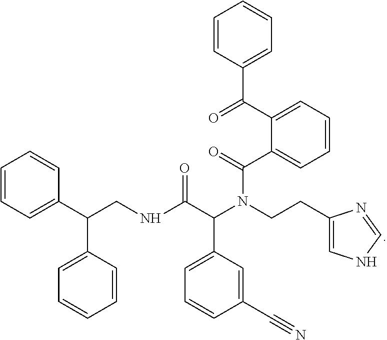 [SMILES: N#Cc1cccc(C(C(=O)NCC(c2ccccc2)c2ccccc2)N(CCc2c[nH]cn2)C(=O)c2ccccc2C(=O)c2ccccc2)c1]

3. The composition of claim 1 wherein the proteasome inhibitor is selected from the group consisting of bortezomib, carfilzomib, MLN 9708, ONX 0912, CEP-1877, NPI-0052, and NLVS.

4. The composition of claim 1 wherein the compound is a ubiquitin-proteasome modulation sensitizing compound and is in an amount effective to potentiate the proteasome inhibitor.

5. A method of potentiating a proteasome inhibitor comprising combining a proteasome inhibitor with a potentiation effective amount of a compound having the following chemical formula: 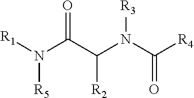
wherein R1 and R5 are each, independently, —H, a substituted or unsubstituted alkyl, a substituted or unsubstituted aryl, substituted or unsubstituted aralkyl or a substituted or unsubstituted heteroaralkyl; R2 is —H, a substituted or unsubstituted alkyl, a substituted or unsubstituted aryl, substituted or unsubstituted aralkyl or a substituted or unsubstituted heteroaralkyl; R3 is a substituted or unsubstituted alkyl, a substituted or unsubstituted aryl, a substituted or unsubstituted heteroaralkyl, or a substituted or unsubstituted heterocycloaralkyl; and R4 is unsubstituted alkyl, a substituted or unsubstituted aryl, substituted or unsubstituted aralkyl, a substituted or unsubstituted cycloalkylalkyl, a substituted or unsubstituted heteroaryl, a substituted or unsubstituted heteroaralkyl, or a substituted or unsubstituted benzophenonyl; or an active derivative thereof,
wherein the compound potentiates the proteasome inhibitor.

6. The method of claim 5 wherein the compound has the following chemical formula:

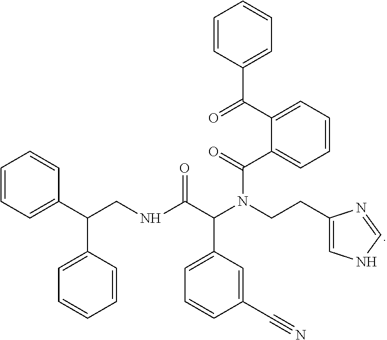 [SMILES: N#Cc1cccc(C(C(=O)NCC(c2ccccc2)c2ccccc2)N(CCc2c[nH]cn2)C(=O)c2ccccc2C(=O)c2ccccc2)c1]

7. The method of claim 5 wherein the proteasome inhibitor is selected from the group consisting of bortezomib, carfilzomib, MLN 9708, ONX 0912, CEP-1877, NPI-0052, and NLVS.

8. The method of claim 5 wherein the compound is a ubiquitin-proteasome modulation sensitizing compound, the proteasome inhibitor and the ubiquitin-proteasome modulation sensitizing compound are administered to a cell or subject, and the compound potentiates a therapeutic effect by the proteasome inhibitor on the subject.

9. The method of claim 8, wherein the subject has cancer and the therapeutic effect is treatment of the cancer.

10. The method of claim 9, wherein the cancer is multiple myeloma.

11. The method of claim 8, wherein the compound is administered concurrently with, after, before or between cycles of administration of the proteasome inhibitor.

12. A method of inhibiting cell proliferation comprising administering to a cell or subject an effective amount of a compound having the following chemical formula: 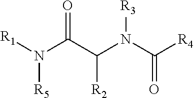
wherein R1 and R5 are each, independently, —H, a substituted or unsubstituted alkyl, a substituted or unsubstituted aryl, substituted or unsubstituted aralkyl or a substituted or unsubstituted heteroaralkyl; R2 is —H, a substituted or unsubstituted alkyl, a substituted or unsubstituted aryl, substituted or unsubstituted aralkyl or a substituted or unsubstituted heteroaralkyl; R3 is a substituted or unsubstituted alkyl, a substituted or unsubstituted aryl, a substituted or unsubstituted heteroaralkyl, or a substituted or unsubstituted heterocycloaralkyl; and R4 is unsubstituted alkyl, a substituted or unsubstituted aryl, substituted or unsubstituted aralkyl, a substituted or unsubstituted cycloalkylalkyl, a substituted or unsubstituted heteroaryl, a substituted or unsubstituted heteroaralkyl, or a substituted or unsubstituted benzophenonyl; or an active derivative thereof,
wherein the compound inhibits cell proliferation in the cell or subject.

13. The composition of claim 12 wherein the compound has the following chemical formula:

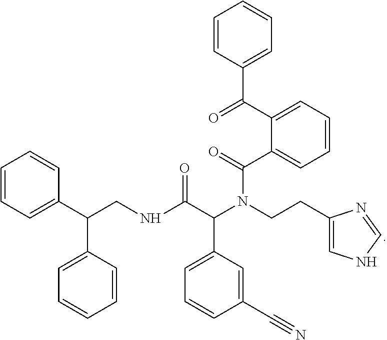 [SMILES: N#Cc1cccc(C(C(=O)NCC(c2ccccc2)c2ccccc2)N(CCc2c[nH]cn2)C(=O)c2ccccc2C(=O)c2ccccc2)c1]

14. The method of claim 12 wherein the compound or active derivative thereof reduces cell cycle progression in the cell or subject.

15. The method of claim 12, wherein the compound is administered concurrently with, after, before or between cycles of administration of a proteasome inhibitor.

16. A method of augmenting glucose utilization comprising administering to a cell or subject an effective amount of a compound having the following chemical formula: 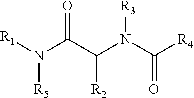
wherein R1 and R5 are each, independently, —H, a substituted or unsubstituted alkyl, a substituted or unsubstituted aryl, substituted or unsubstituted aralkyl or a substituted or unsubstituted heteroaralkyl; R2 is —H, a substituted or unsubstituted alkyl, a substituted or unsubstituted aryl, substituted or unsubstituted aralkyl or a substituted or unsubstituted heteroaralkyl; R3 is a substituted or unsubstituted alkyl, a substituted or unsubstituted aryl, a substituted or unsubstituted heteroaralkyl, or a substituted or unsubstituted heterocycloaralkyl; and R4 is unsubstituted alkyl, a substituted or unsubstituted aryl, substituted or unsubstituted aralkyl, a substituted or unsubstituted cycloalkylalkyl, a substituted or unsubstituted heteroaryl, a substituted or unsubstituted heteroaralkyl, or a substituted or unsubstituted benzophenonyl; or an active derivative thereof,
wherein the compound augments glucose utilization of the cell or subject.

17. The composition of claim 16 wherein the compound has the following chemical formula:

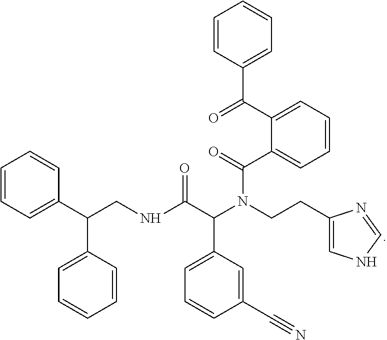 [SMILES: N#Cc1cccc(C(C(=O)NCC(c2ccccc2)c2ccccc2)N(CCc2c[nH]cn2)C(=O)c2ccccc2C(=O)c2ccccc2)c1]

18. The method of claim 16, wherein the compound is administered concurrently with, after, before or between cycles of administration of a proteasome inhibitor.
